(19) United States
(12) Patent Application Publication
(10) Pub. No.: US 2016/0081712 A1
(43) Pub. Date: Mar. 24, 2016

(54) DEVICE, SYSTEM, AND METHOD FOR INSERTION OF A MEDICAL DEVICE INTO A SUBJECT

(71) Applicant: THE CHARLTTE-MECKLENBURG HOSPITAL AUTHORITY D/B/A CAROLINAS HEALTHCARE SYSTEM, Charlotte, NC (US)

(72) Inventors: B. Todd Heniford, Charlotte, NC (US); Amy E. Lincourt, Charlotte, NC (US)

(21) Appl. No.: 14/786,802

(22) PCT Filed: Apr. 29, 2014
(86) PCT No.: PCT/US14/35845

§ 371 Date: Oct. 23, 2015

Related U.S. Application Data
(60) Provisional application No. 61/817,032, filed on Apr. 29, 2013.

(51) Int. Cl. A61B 17/34

(52) U.S. Cl. CPC A61B 17/3403; A61B 17/3474; A61B 2019/5206

(57) ABSTRACT

A penetrator device (12), system (10), and associated method are provided that may help inform an operator of the position of a penetrating edge of the penetrator device within a subject. In one aspect, a penetrator device, such as a needle (12a), probe, cannula, or the like, includes an optical imaging system (14) that is configured to identify the position of a penetrating end of the penetrator device in a subject based on optical data received by the optical imaging system. The system is configured to identify the position of the penetrator device and provide a signal that alerts the operator to the position of the device within the subject.

### BACKGROUND OF THE INVENTION

There are many circumstances where it may be desirable to insert a medical device, such as a probe or needle, into a patient. Examples of such procedures include laparoscopy, thoracoscopy, mediastinoscopy, and insertion of a catheter into a lumen of an anatomical conduit, such as a blood vessel.

In laparoscopy, one or more small incisions (e.g., 0.5 to 1.5 cm) are created in the abdominal wall of a patient to provide access to the patient's abdominal cavity. Typically, an imaging device, such as a laparoscope, is inserted into the abdominal cavity through one of the incisions to allow the surgeon to view the operation site.

In some laparoscopic procedures, the abdominal cavity is initially insufflated with a gas, such as carbon dioxide. Insufflation causes distension and elevation of the abdominal cavity above the internal organs to create a working and viewing space which helps facilitate examination and manipulation of the cavity contents. Insufflation is typically performed with a specialized needle called a Veress needle. The Veress needle includes an outer cannula having a beveled needle point for cutting through various tissue layers of the abdominal wall. Many conventional Veress needles include a spring-loaded, inner stylet that is positioned within the outer cannula of the needle. This inner stylet typically comprises a polymeric material and includes a dull tip that functions to protect any internal organs from injury by the needle end of the Veress needle. Penetration of the needle through the abdominal wall causes the stylet to be pushed inside the outer cannula. When the tip of the needle enters a space such as the peritoneal cavity, the stylet springs forward to help prevent the needle end of the Veress needle from contacting any internal organs. An insufflation gas, for example, carbon dioxide, is then passed through the Veress needle to inflate the abdominal cavity.

Since laparoscopy utilizes one or more small incisions, it may provide many benefits over an open access procedure. In particular, laparoscopic surgery can typically be performed with lower incidences of hernias and wound complications. In addition, the small incisions associated with laparoscopic surgery are more cosmetically appealing in comparison to an open access incision.

Laparoscopy also has some disadvantages. For example, during penetration of the Veress needle into the abdominal cavity, a surgeon must determine the progress of insertion of the needle end through the various tissue layers of the abdominal cavity. However, the surgeon generally has little information for determining the position of the end of the needle during penetration, and typically needs to rely on the detection of sound as the needle end penetrates, and/or the utilization of touch and feel of the physical resistance, or lack of resistance, against the needle end during penetration.

An additional prior technique includes measuring changes in pressure maintained at the penetrating end of a Veress needle during penetration of the multiple layers of the umbilical region of the abdomen. The multiple layers of the umbilical region include the outer skin layer, a fat cell layer of variable thickness, a fascia layer of variable tissue thickness and abdominal muscles, a peritoneum layer, and the abdominal cavity. Each of the layers of the umbilical region may vary in depth between patients, and there may be other complicating factors, such as the presence of scar tissue. Accordingly, it is often difficult to determine the position of the needle using this technique as well.

Improper positioning of the needle may result in perforation of an internal organ or injury to retroperitoneal vessels. In addition, improper determination of the needle's position may result in injecting carbon dioxide into the abdominal wall. As a consequence, the penetration of a needle or a similar probe during the insufflation technique requires an extremely delicate sequence of steps, which normally needs to be performed by an experienced surgeon.

Accordingly, there still exists a need for improved devices, systems, and methods for the insertion of medical devices into a patient.

### BRIEF SUMMARY OF THE INVENTION

Aspects of the present invention are directed to a penetrator device, system, and associated method that may help to address one or more of the problems associated with the prior art. In particular, aspects of the present invention are directed to a penetrator device, such as a needle, probe, cannula, or the like, that includes an optical imaging system that is configured to identify the position of a penetrating end of the penetrator device in a subject based on optical data received by the optical imaging system. In some aspects, systems in accordance with the present invention are configured to identify the position of the penetrator device and provide a signal that alerts the operator to the position of the needle within the subject.

With countless medical procedures requiring insertion of devices into a patient, improving this process would provide a much needed benefit for patients and providers. For instance, in laparoscopy, the insertion of a Veress needle is a procedure that has not been improved upon in decades. As discussed previously, insertion of the needle into a patient is essentially a blind process that has potential devastating complications of injury to intestines or other internal organs, as well as hemorrhage from major blood vessels. Only sound and feel of the needle's insertion along with surgeon experience guide the current process. This process can also take additional time as it is not uncommon to require multiple attempts at insertion. Aspects of the present invention may help overcome these problems by utilizing an optical imaging system, such as an optical coherence tomography (OCT) imaging system, by providing the operator a signal, such as a visual or auditory signal, when the penetrating end of the needle is in a desired location in the subject. In particular, aspects of the present invention may help operators, such as surgeons, to have information regarding the position of the needle in the subject. As a result, aspects of the invention may help to decrease the risk of injury and harm to the patient. In addition, aspects of the invention may also help to improve the speed of the overall procedure.

In aspect, a penetrator device in accordance with the present invention comprises an elongate member having a distal portion and a proximal portion defining a cannula body, and a longitudinally extending conduit that extends from the proximal portion of the elongate member to an opening disposed towards the distal portion of the elongate member. A penetrating edge, such as a sharpened edge or tip, is disposed at the distal portion of the elongate member. An optical device is disposed in the conduit and includes an optical lens that is configured to receive optical data of the tissue of the subject. In one embodiment, the optical device being configured to receive optical data of the subject through said opening and to communicate the optical data to an associated processor that is configured to identify the position of the penetrator device within the subject based on the optical data.

In one embodiment, the optical device includes an optical lens and an associated optical fiber that is configured to be connected an in communication with an optical imaging system. In one embodiment, the optical imaging system comprises an OTC system.

In one aspect, the optical device comprises a longitudinally extending cannula having a distal portion and a proximal portion, and an optical lens that is disposed in the cannula towards the distal portion thereof. A fiber optic cable that is in communication with the optical lens is configured to communicate optical data from the optical to lens to an associated processor.

In some embodiments, the penetrator device may also include one or more additional channels that are configured to introduce a gas, such as CO2 into the subject during insertion of the penetrator device. In one embodiment, the one or more channels are disposed in the elongate member and extend longitudinally from the proximal to the distal portion of the elongate member. In aspect, the one or more channels define an annular channel that is disposed between the optical device and an inner surface of the elongate member of the penetrator device. The one or more channels are typically in communication with a gas source and define a fluid pathway for the gas from the gas source to the opening of the penetrator device.

In one aspect, the invention also provides a system for inserting a medical device into a subject. In one embodiment, the system comprises a penetrator device having an elongate member having a distal portion and a proximal portion, a longitudinally extending conduit that extends from the proximal portion of the elongate member to an opening disposed at the distal portion of the elongate member, a penetrating edge disposed at the distal portion of the elongate member, and an optical device disposed in the conduit.

The optical device may be configured to receive optical data of the subject through said opening and communicate the optical data to an optical imaging system in communication with the optical device.

In some embodiments, a fiber optic cable may be used to communicate optical data between the optical imaging system and the optical device.

In one aspect, the imaging system may include a light source for emitting light to the optical device, a fiber optical cable disposed between the optical device and the optical imaging system, and a computer and/or processor configured to analyze optical data reflected back to the optical imaging system and identify the position of the penetrator device within the subject based on the optical data.

The system may include one or more modules, such as a program module that is configured to communicate a signal to the operator based on the position of the penetrator device in the subject. In one aspect, the system includes one or more modules that are configured to perform one or more of the following: emit a signal that communicates the position of the device within the subject; emit a signal that alerts an operator when the device is in a desired position within the subject; analyze the intensity of light reflected back to the optical imaging system and determine if the penetrating edge of the penetrator device has entered the peritoneal cavity of the subject; communicate a signal to instruct an operator to abort or stop the procedure; detect an index of refraction mismatch in light emitted by the light source; emit a signal that identifies the tissue adjacent to the penetrating edge of the penetrator device.

In one aspect, the signal generated by the system may be visual, auditory, or a combination thereof. In one embodiment, the signal generated by the system may comprise a light indication that communicates to the operator to continue the procedure, such as to continue to insert the penetrator device into a subject. In some embodiments, the signal generated by the system may comprise a light indication that communicates to the operator to stop insertion of the penetrator device, or to abort the procedure.

Aspects of the present invention are also directed to a method of inserting a medical device, such as penetrator device into a subject. In one embodiment, the method my comprise the steps of inserting a medical device into one or more tissue layers of a subject; collecting optical data of the one or more tissue layers; analyzing the optical data to determine a position of a penetrating end of the medical device in the subject; and communicating a signal to an operator identifying the position of the penetrating end of the medical device in the subject.

In one embodiment, the step of communicating a signal comprises generating a visual or auditor signal. In a further aspect, may include the step of generating a signal that alerts the operator if the penetrating end of the medical device is in a desired position in the subject, or the step of generating a signal that alerts the operator if the penetrating end of the medical device is in an undesired position in the subject.

In one aspect, the method may include the step of generating a signal that alerts the operator to continue the step of inserting the medical device. In another aspect, the method may include the step of generating a signal that alerts the operator to abort or stop of inserting the medical device.

In one embodiment, the step of communicating a signal comprises generating a green light to alert the operator to continue to insert the medical device. In one embodiment, the step of communicating a signal comprises generating a red light to alert the operator to stop the step of inserting the medical device. In some aspects, the step of communicating a signal further comprises identifying a tissue type of the subject that is adjacent to the penetrating end of the medical device.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 illustrates a schematic illustration of an example of a system in accordance with some embodiments discussed herein;

FIG. 2 is a side view of a penetrator device in accordance with some embodiments of the present invention;

FIG. 3 is a side view of a imaging device in accordance with some embodiments of the present invention;

FIGS. 4A and 4B illustrate an embodiment of a penetrator device in accordance with some embodiments of the present invention;

FIG. 5 is a schematic illustration of an example of a system in accordance with some embodiments discussed herein;

FIG. 6 is an illustration of a flow chart in accordance with an embodiment of the present invention

FIG. 7 includes two charts that show expected back reflectance as a function of tissue depth;

FIG. 8 is an illustration of a flow chart in accordance with an embodiment of the present invention; and

FIG. 9 illustrates a block diagram of circuitry which may be included in an imaging system according to an example embodiment.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

As used herein, the terms “data,” “content,” “information” and similar terms may be used interchangeably to refer to data capable of being captured, transmitted, received, displayed and/or stored in accordance with various example embodiments. Thus, use of any such terms should not be taken to limit the spirit and scope of the disclosure. Further, where a computing device is described herein to receive data from another computing device, it will be appreciated that the data may be received directly from the another computing device or may be received indirectly via one or more intermediary computing devices, such as, for example, one or more servers, relays, routers, network access points, base stations, and/or the like. Similarly, where a computing device is described herein to send data to another computing device, it will be appreciated that the data may be sent directly to the another computing device or may be sent indirectly via one or more intermediary computing devices, such as, for example, one or more servers, relays, routers, network access points, base stations, and/or the like.

The principles described herein may be embodied in many different forms. Not all of the depicted components may be required, however, and some implementations may include additional, different, or fewer components. Variations in the arrangement and type of the components may be made without departing from the spirit or scope of the claims as set forth herein. Additional, different, or fewer components may be provided.

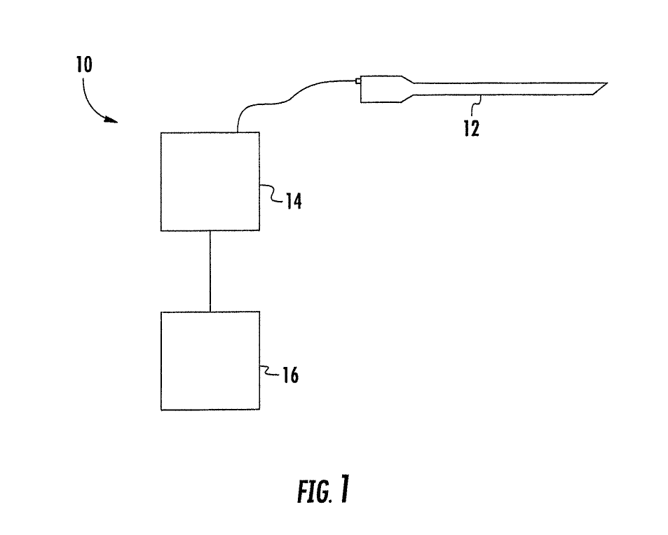

With reference to FIG. 1, a system in accordance with at least one aspect of the present invention is illustrated and represented by reference character 10. System 10 includes a penetrating device 12, an optical imaging system 14, and an associated computer or processor 16. The penetrator device, which includes a sharpened tip defining a penetrating end of the penetrator device, is configured to be inserted into and through one or more tissue layers to a desired location within a subject. In one aspect, the optical imaging system includes an optical device that is disposed in an interior space of the penetrating device and is configured to receive optical data of the subject that is then analyzed by the optical imaging system and associated computer.

Based on the analysis of the optical data, the system 10 is configured to generate a signal, such as auditory or visual signal, that alerts an operator of the position of the medical device within the subject.

For example, in the case of a laparoscopic procedure, the system may be configured to generate a signal when the penetrating end of the medical device has entered the peritoneal cavity (also referred to as the abdominal cavity) of the subject. In this way, the operator is alerted to the position of the medical device so that improper positioning of the medical device may be avoided.

In the present invention, the term “subject” includes mammals, birds, fish, reptiles, amphibians, and the like. In a preferred embodiment, the subject is a mammal, such as a human.

Penetrator device 12 may comprise any device that is configured to pierce and be inserted into one or more material layers of a subject. For example, penetrator device 12 may comprise a needle, cannula, probe, or the like.

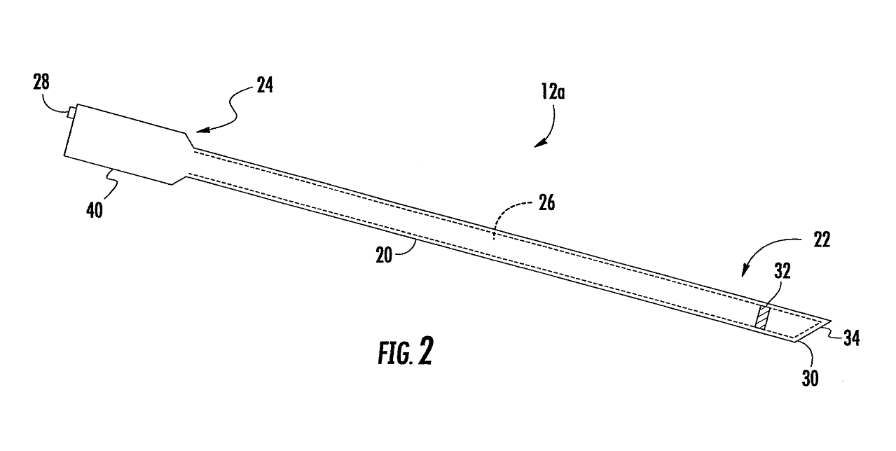

In this regard, FIG. 2 illustrates an embodiment of the invention in which the penetrator device comprises a needle 12a. In one aspect, needle 12a comprises an outer cannula having a shape for piercing materials, such as tissues, organs, and walls of blood vessels. Needle 12a comprises an elongate member 20 having a distal portion 22 and a proximal portion 24. The interior of the elongate member 20 includes a longitudinally extending conduit 26 (represented by the dashed lines in FIG. 2) that extends from a port 28 located towards the proximal portion 24 of the elongate member 20 and an opening 30 disposed at the distal portion 22 of the elongate member 12. In one embodiment, an optical device 32 is disposed in the conduit and is configured to receive optical data of the subject through opening 30 of the needle 12a. Optical device 32 is typically in communication with optical imaging system (see reference character 14, FIG. 1) via an optical fiber (not shown) that extends through the conduit to optical port 28.

The distal portion 22 includes a pointed edge or sharp edge defining the penetrating end 34 of the needle 12a. In one embodiment, the penetrating end 34 is sharply pointed and has a shape similar to that in conventional trocars. The elongate member 20 of the needle 12a is preferably constructed of a material that is rigid enough to be inserted into the desired material of the subject. Suitable materials for the elongate member may include a metallic alloy, such as stainless steel, plastic, and other polymers.

The needle typically has a small diameter, for example, from about 0.25 to 2.5 mm, and in particular, from about 0.75 to 1 mm yet generally large enough to accommodate the smallest diameter optical fiber. The overall length of the elongate member is desirably sufficient so that it can be inserted and directed to a desired location in a subject. For example, in applications directed to laparoscopic surgery, the length of the elongate member may range from about 3 to 12 centimeters (cm), and in particular, about 5 to 10 cm.

In a further aspect of the invention, the needle 12a may include a handle assembly 40. In a preferred embodiment of the invention, the needle 12a may be held in a manner similar to a pencil and inserted into the biological tissue or material that is to be imaged. However, without loss of generality, it is also understood that the needle 12a can be held by other means.

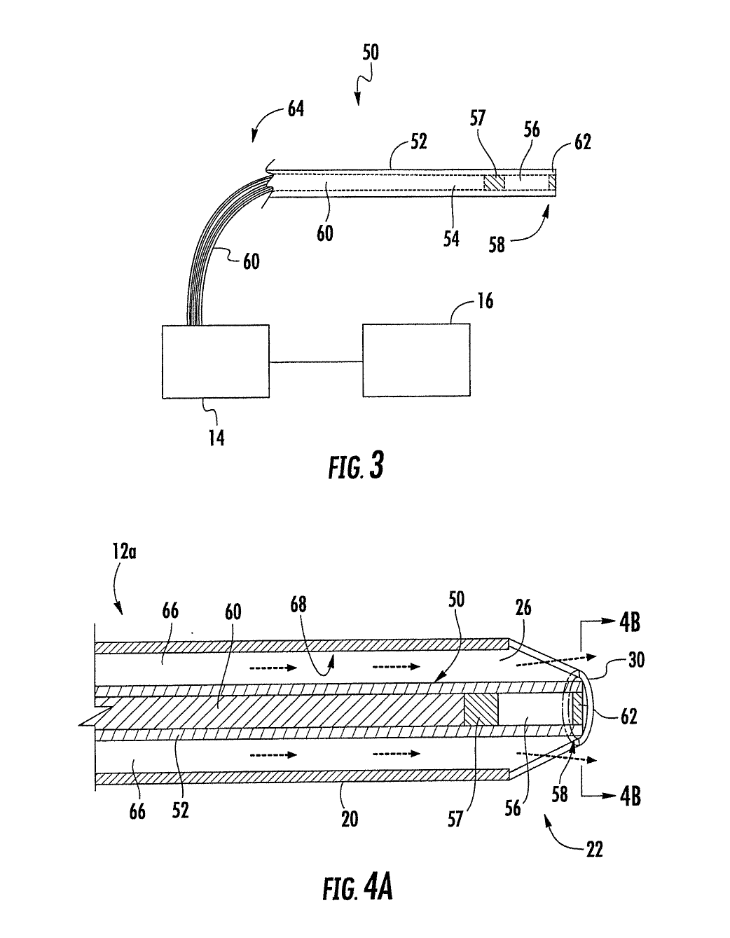

Turning now to FIG. 3, an example of a representative optical device 32 that may be used in aspects of the present invention is illustrated and broadly designated by reference number 50. As noted above, optical device 50 is configured to be disposed in the conduit (see FIG. 2, reference character 26) of the penetrator device 12. In one embodiment, the optical device includes a cannula 52 defining an interior bore 54 in which the components of the optical device are disposed. The cannula typically comprises a rigid or semi-rigid material such as a metal (e.g., stainless steel) or polymeric material.

In one embodiment, the optical device 50 may be in a fixed position within the conduit 26. In other embodiments, the optical device 50 may be removable from the conduit. For example, in some aspects the optical device 50 may be removed during a procedure to allow access through the conduit 26 of the penetrator device.

The optical device includes an optical lens 56 disposed towards a distal portion 58 of the cannula, an index matching material 57, such as a gel, disposed on the proximal side of the optical lens 56, and a fiber optic cable 60 that extends from the optical lens to a proximal portion 64 of the cannula where it exits the penetrator device via optical port 28. In some embodiments, the fiber optic cable 60 within the penetrator device may terminate at the optical port 28. For example, fiber optical cable 60 may be connected to the optical port with a standard connector, such as an “FC” type connector. Optical port may then be connected to a longer fiber optical cable that is connected to optical imaging system 14 (for example, a light source and an optical time domain reflectometer). In one embodiment, the optical device is connected directly to the optical imaging system, and in other embodiments, one or more optical fibers are connected in series to connect the optical device 50 to the imaging system.

In some aspects of the invention, the optical lens may comprise a GRadient INdex (GRIN) lens. In the illustrated embodiment, a reflective coating 62 may be present on the distal end of the optical lens. Preferably, the reflective coating is disposed on the outer surface of the optical lens, and is partially reflected to reflect a portion of the light (e.g., from about 5 to 10%) emitted by a light source back to the imaging system. In particular, the reflective coating may be present to provide a reference beam that is reflected back to the imaging system, such as to an interferometer. Alternatively, the optical device 50 may include a small air gap that can also be used to provide a reference beam. For example, the optical device may include a small transparent cap, such as glass or transparent plastic material at the distal end of the conduit and a small air gap between the cap and optical lens. Preferably, the size of the air gap would be relatively small, for example, about 1 mm or less.

The penetrator device can be used in conjunction with a number of different types of optical imaging systems, in particular, with systems which deliver and collect a single spatial mode optical beam. In one embodiment, the optical device and associated imaging system comprise an optical coherence tomography (OCT) imaging system. There are a variety of OCT imaging systems which are included within the scope of the invention, including those which provide optical path length scanning, tunable optical source scanning, optical source scanning, optical spectrum analysis imaging, and optical phase delay-line scanning. Other interferometric imaging and ranging techniques are also encompassed within the scope of the present invention. OCT is the preferred imaging technology to be used with the penetrator device described herein because it can perform very high sensitivity and high dynamic range measurements of the echo time delay and intensity of back reflected and backscattered light.

In one aspect of the invention, the fiber optic needle probe communicates with the imaging engine of an OCT device by means of a single mode optical fiber that is at least partially housed within the penetrator device. In particular, at least a portion of an optical fiber 60 is positioned within a bore defined by cannula. Suitable materials for optical fiber 60 may include glass, plastic, and other suitably optically transparent materials. In a preferred embodiment of the invention, the optical fiber 60 is a single mode optical fiber 60 which emits and collects a single, or nearly single, transverse mode optical beam at, or near, its distal end. The fiber 60 typically comprises a core, usually cylindrical in profile, of elevated index of refraction (e.g., glass) surrounded by a cladding. The diameter of the core typically depends upon the wavelength of the light that it is designed to carry as well as the optical properties of the core and the cladding. For example, the diameter of the core is generally in the range of 4 to 12 μm.

The cladding of the fiber 60 desirably has a diameter large enough such that the electric field of the optical beam or mode which is present in the cladding is not substantially perturbed by the outer boundary of the cladding so as to introduce appreciable loss or dispersion, since the optical beam or mode in the core decays in an exponential manner in the cladding as a function of the distance away from the core. The cladding surrounds the core of the fiber 60 and the typical cladding diameter in a fiber 60 used for optical communications is about 125-200 microns. However, it is understood that while the aforementioned are typical dimensions of the cladding, the cladding can have a wide range of diameters and can be as small as 12-15 μm. Accordingly, extremely small optical fiber 60 diameters can be constructed which carry single mode optical beams.

In a further embodiment of the invention, the optical fiber 60 is provided with a coating or jacket surrounding the cladding, which can be a plastic, metal, polymer material, or the like. The jacket of the fiber 60 does not serve to guide the optical beam but acts to protect the fiber 60, strengthen it from breaking, and to increase its mechanical rigidity. In an embodiment of the invention where the fiber 60 is integrated with the penetrator device 12, the penetrator device 12 casing itself may serve as the jacket.

With reference to FIGS. 4A and 4B, an embodiment of the penetrator device is shown in which the needle 12a is particularly useful in laparoscopic procedures. FIG. 4A shows a partial view of an embodiment of the invention in which the needle 12a includes elongate conduit 26 having the optical device 50 disposed therein. FIG. 4B is a front cross-sectional view of the penetrator device taken along lines 4B of FIG. 4A.

In this embodiment, the elongate conduit 26 includes an annular channel 66 disposed between optical device 50 and the inner surface 68 of the conduit 26. Annular channel provides a fluid pathway between a gas source (not shown) and the opening 30 of the needle 12a. During penetration of the penetrating end of the needle 12a into a subject, a gas, such as CO₂, is introduced into the annular channel. The gas (represented by the arrows in FIG. 4A) then flows through the annular channel where it is introduced into the subject via opening 30. Preferably, the gas is introduced a minimal flow rate (e.g., from about 5 to 500 mL/min., and in particular, from about 25 to 100 mL/min.) to assist in separation of adjacent tissue layers. It has been found that obtaining such a separation assists the optical device in acquiring optical data that can be used to ascertain the position of the needle in the subject.

As discussed in greater detail below, the optical device 50 captures optical data that is then transmitted to the optical imaging system. The optical system (which in some embodiments may include a light source and an optical time domain reflectometer (OTDR)) is then configured to communicate the information contained in the optical data to a computer or associated processor. The computer/processor includes one or more modules that are configured to analyze the information and provide real-time information regarding the position of the penetrator device in the subject. In this way, the operator can be instantly alerted to the position penetrator device.

The penetrator device is designed for use in conjunction with any optical imaging system which requires the controlled delivery and collection of a single spatial mode optical beam. The imaging system typically includes the associated sub-systems such as optics, electronics, motors, computers, and controls necessary to generate high resolution images, control image acquisition, or otherwise, process, quantitate, and display images. Although the penetrator device of the present invention can be integrated with OCT, it is also understood that cross-sectional images can be measured using any technique which is capable of performing a high resolution, high sensitivity measurement of the echo time delay, coherence properties, frequency properties, or other properties, of backreflected or backscattered light signals. These techniques include, but are not limited to, nonlinear cross-correlation techniques which measure the time variation and intensity of light. It is understood that aspects of the invention may be applied with any optical measurement diagnostic technique (i.e., both imaging and nonimaging) which requires the delivery of a single transverse mode optical beam and the collection of reflected, remitted, or backscattered light in a single spatial optical mode where the position or orientation of the optical beam is scanned in a controlled pattern. These include imaging modalities such as other interferometric and noninterferometric imaging systems, fluorescence and other spectroscopic imaging systems, Raman imaging, single photon confocal imaging, multiphoton confocal imaging systems, and combinations thereof. It is also understood that the penetrator device may be used in a nonimaging modality where it is desired to perform optical measurements of internal body structures on a microstructural scale, but where data may be represented in a form other than an image.

There are several different embodiments of OCT imaging systems including: (1) embodiments which use a low coherence light source and an interferometer in conjunction with methods which scan the group and phase delays of light in a reference arm or shift the frequency of light in a reference arm; (2) embodiments which use a low coherence source, an interferometer, and an optical spectrum analyzer to analyze the output spectrum of the interferometer; and (3) embodiments which use a narrow linewidth frequency tunable optical source and interferometer. In addition, there are other systems which use non-interferometric methods to measure the echo time delay of backscattered or backreflected light. It is also understood that the application of the penetrator device is not limited to OCT imaging.

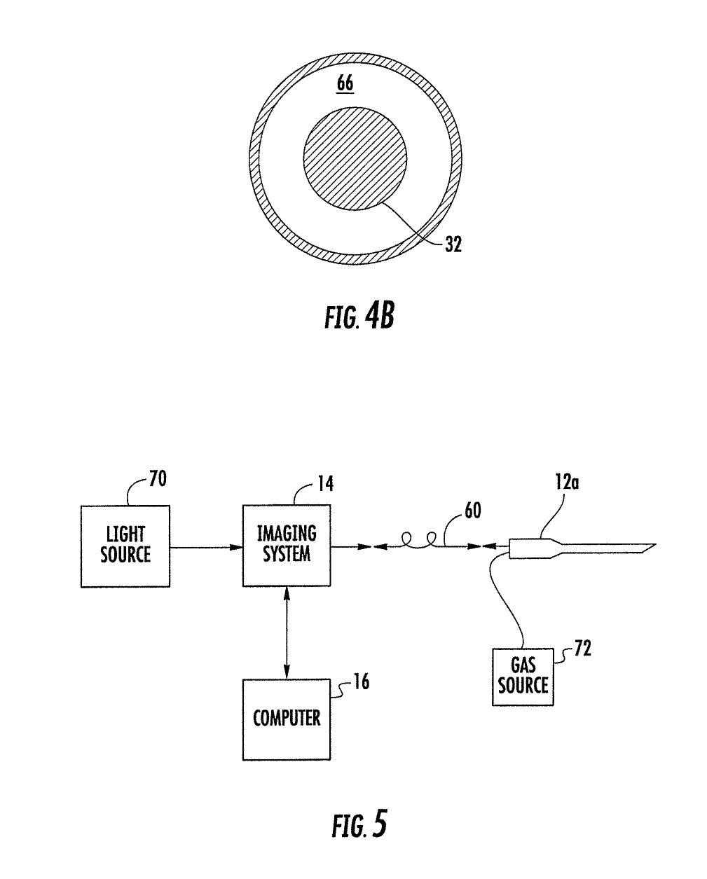

Referring to FIG. 5, a schematic containing the principle system modules in an OCT imaging system used with the penetrator device in a preferred embodiment of the invention is shown. The system includes the penetrator device module which consists of a needle 12a, optical fiber 60. Light is coupled from an optical source 70 to the optical fiber 60 in the needle 12a and directed into the tissue or other specimen which is to be measured or imaged. An OCT imaging system 14 performs high sensitivity, high precision measurements of echo time delay (distance) and magnitude of the backscattered or backreflected light through the optical fiber 60. The resulting data is transmitted to a computer/processor 16 where it is analyzed to determine position of the needle in the subject. In some embodiments, the data may also be used to generate a video signal such as NTSC or PAL signal and the data directly displayed on a display device, such as a TV monitor. In a preferred embodiment, the computer processes this information and represents it as an image on a display device.

In one embodiment, the light source comprises a low coherence light source, such as a superluminescent diode laser. The wavelength of the light is not critical to most aspects of the invention, and therefore a wide variety of light sources may be used in various aspects of the invention. In one embodiment, the light source uses approximately 1310 nm center wavelength in the near-infrared spectrum. Other light sources, for example, at 800, 1060, and 1550 nm may also be used in embodiments of the invention. In some embodiments, the light source may comprise one or more of the following types of light sources: superluminescent diode laser, Semiconductor Optical Amplifiers (SOA), Frequency Swept Laser Sources, Ultrashort Pulsed Lasers, Supercontinuum Lasers, and Amplified Spontaneous Emission (ASE) light sources.

FIG. 5 also shows an optional gas source 72 that may be present in certain embodiments of the invention. In particular, gas source 72 may be present to provide a flow of CO₂ during the process of inserting the penetrator device into a subject.

Although FIG. 5 shows the imaging system 14 and the computer 16 as separated modules/devices, it should be recognized that each of these modules/devices may be incorporated into a single device, or alternatively, comprise separate devices that are in communication with each other.

Optical data received by the optical imaging system is communicated to the computer/processor for further analysis. The computer includes one or more modules that are configured to analyze the data and determine the position of the penetrating end of the penetrator device within the subject.

In one embodiment, the computer is configured to detect a change in the index of refraction of the light as it moves from one type of tissue to a different type. For example, in a laparoscopic procedure, the computer is configured to analyze the intensity of the back reflected light that is transmitted through the optical fiber. As the penetrating end of the penetrator device enters the peritoneal cavity, the imaging system will detect a change in the index of refraction, which will provide a strong reflection peak followed by no signal. The computer analyzes the data and is configured to generate a signal to an operator that the penetrator device is in a desired location within the subject.

For medical applications, the operator inserts the penetrator device into the tissue being imaged. In one embodiment of the invention, the imaging process is ongoing during the insertion procedure, in which case, this image information is used to guide the insertion of the device and its placement. In a further embodiment of the invention, penetrator device insertion and placement is guided using external features and landmarks of the body and known anatomy. Insertion and placement may also be guided based on data from other medical diagnostic modalities such as X-ray, computed tomography, ultrasound, magnetic resonance imaging, and the like. Where applicable, other forms of imaging including microscopic, laparoscopic, or endoscopic visualization; ultrasound, magnetic resonance imaging, radiography or computed tomography may be performed prior to, or in real-time, during the insertion and placement of the device.

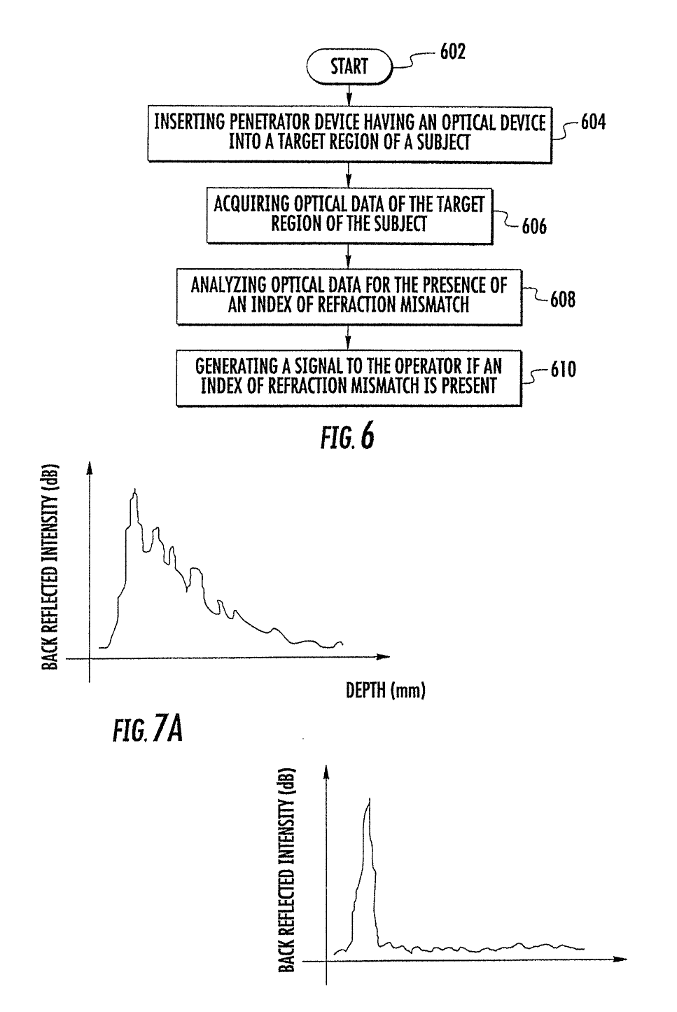

FIG. 6 illustrates a flow chart of an example embodiment of a method of inserting a penetrator device in the body of a subject. Method 600 may begin at 602 and proceed to 604 where an operator begins insertion of a penetrator device having an optical device into a patient. For example, in the case of a laparoscopic procedure, the operator will begin insertion of the penetrating end of the penetrator device (e.g. needle 12a) through layers of tissue covering the umbilical region.

At 606, the optical imaging system acquires optical data via the optical lens that is transmitted back through the optical fiber. Generally, the optical data is acquired and transmitted in a continuous or semi-continuous manner to the optical imaging system so that analysis of the data can be performed in real-time as the procedure progresses.

At 608, the optical data is analyzed by the computer for the presence of an index of refraction mismatch. As noted above, the presence of an index of refraction mismatch will generally result in a relatively strong reflection peak followed by no signal. The presence of the mismatch will alert the computer that the penetrating end has arrived at a desired location within the subject. In one aspect of the invention, the analysis may include comparing the intensity of the optical data captured from within the subject to the intensity of a reference beam that is generated by the optical lens and transmitted back to the associated optical imaging system.

At step 610, the computer is configured to generate a signal that will alert the operator that the penetrator device is in a desired location so that further penetration of the penetrator device is immediately halted. In some embodiments, the step of generating a signal will be performed by transmitting a signal to the operator to stop further penetration of the penetrator device. For example, the computer may be configured to generate a visual or auditory signal that alerts the operator to stop further penetration. In one embodiment, a visual display may be configured to generate a visual display such as a “red light” that alerts the operator to stop insertion of the penetrator device. In other embodiments, the computer may transmit instructions to an auditory device, such as a speaker, to produce an auditory signal that alerts the operator to stop penetration of the penetrator device. In one aspect, the computer may be configured to generate a visual or auditory signal that alerts the operator to continue insertion of the penetrator device in the patient, such as the generation of a green light.

In one embodiment, the computer is configured to generate a green light that will alert the operator to proceed and continue with the insertion of the penetrator device in the subject. Upon analysis of optical data that the penetrating end is at a desired location in the subject, the computer will then generate a signal to alert the operator that further insertion of the penetrator device is not needed.

In a further aspect of the invention, the computer may be configured to generate a signal to the operator to abort or restart the procedure. For instance, the computer may be configured to include one or more modules that will alert the operator if the penetrating end of the penetrator device has been positioned in an undesirable location within the patient. In one such aspect, the computer may be configured to transmit a signal, in the form of a sound or visual indication, that the procedure should be aborted and/or the penetrator device repositioned within the subject. Such a signal could be based, for example, audible signal, text or visual image on a display device, on a color light indication or a flashing light indication, such as a blinking red light, or a combination thereof.

In a further embodiment, the method may also include introducing a minimal gas flow through the penetrator device during insertion of the device into a subject. As discussed previously, the gas may be used to separate adjacent tissue layers to assist the optical lens in obtaining image data within the subject. In particular, in a laparoscopic procedure, the flow of gas may help separate the inner tissue layer in the peritoneal cavity so that an index of refraction mismatch can be detected by the system. In this regard, FIGS. 7A and 7B illustrate charts that depict the expected back reflected intensity during penetration of the needle into the subject. As shown in FIG. 7A, it is expected the back reflectance will gradually decline with increasing tissue depth. However, upon entrance of the penetrating tip of the needle into the peritoneal cavity, it is expected that the CO₂ gas will create a tissue separation that permits the optical system to detect a glass (n=1.5) to air (n=1) index of refraction mismatch that is characterized by a strong reflection peak followed by substantially no signal.

Generally, the gas is introduced at a rate that is between 1 L/min and 40 L/min, and in particular, between 5 L/min and 10 L/min.

Once the operator has received a signal that the penetrator device is in a desired location, the operator can then begin introducing an insufflation gas into the subject in order to insufflate the body cavity, such as the peritoneal cavity.

In other embodiments, the computer may be configured to analyze optical images obtained of the tissue, and based on these images, determine the position of the device within the subject. For example, the computer may include one or more program and/or modules that are configured to differentiate and recognize specific types of tissue. As a result, the computer will then know in real-time the precise location of the penetrating end of the penetrator device within the subject. As in the method described in FIG. 6, above, the computer may be configured to generate a signal that will alert the operator that the penetrating end is in a desired location so that further penetration of the penetrator device is immediately halted.

In still further aspects of the invention, the computer may be configured to recognize specific tissue types of a subject and send a corresponding signal to the operator depending on the tissue. In one embodiment, the computer may be configured to analyze and identify specific tissue types based on the analysis of the optical data received from the optical device. As a result of this analysis, the computer may be configured to communicate the tissue type to the operator so that the operator is informed of the position of the penetrating end of the device. For example, based on the optical data, such as optical images of the tissue, the computer may be configured to transmit the location of the penetrating end to the operator of the system. In this way, the operator can be informed of the position of the device within the subject.

In the case of a laparoscopic procedure, the computer may be configured to analyze the optical data and identify specific tissue, such as whether the penetrating end is still in the wall of the abdominal cavity, whether the penetrating end has entered the peritoneal cavity of the subject, and whether the penetrating end has entered an undesirable location within the subject, such as the intestine or organ (e.g., liver, spleen, etc.). In some embodiments, the system may be configured to generate a specific alert to the operator depending on the tissue identified by the computer. For example, the computer upon receiving and analyzing optical data which identifies that the penetrating end of the penetrator device has entered the intestinal wall may be configured to communicate to the operator that the intestine has been perforated. The operator being thus apprised of this information will be able to take the appropriate action.

FIG. 8 illustrates a flow chart of a further example embodiment of a method of inserting a penetrator device in the body of a subject. Method 800 may begin at 802 and proceed to 804 where an operator begins insertion of a penetrator device having an optical device into a patient. For example, in the case of a laparoscopic procedure, the operator will begin insertion of the penetrating end of the penetrator device through layers of tissue covering the umbilical region.

At 806, the optical imaging system acquires optical data via the optical lens that is transmitted back through the optical fiber. Generally, the optical data is acquired and transmitted in a continuous or semi-continuous manner to the optical imaging system so that analysis of the data can be performed in real-time as the procedure progresses.

At 808, the optical data is analyzed by the computer to determine the type of tissue of subject that is being encountered by the penetrating end of the penetrator device. As noted above, the computer may be configured to include one or more modules that analyze the optical data to determine the specific tissue type being imaged by the optical device of the penetrator device. In one aspect, the step of analyzing the optical data may include the step of comparing the optical data of the subject to stored optical data of specific tissue types. In this way, the computer may be configured to identify the tissue type being imaged by the optical device of the penetrator device.

At step 810, the computer is configured to generate a signal that will alert the operator depending on the tissue type of the subject being imaged by the penetrator device. For example, if the penetrating end has reached a position where it is in a desired location, the computer may be configured to generate a signal to instruct the operator to halt further penetration of the penetrator device. In other embodiments, the computer may be configured to generate a signal to the operator to continue to proceed with the insertion of the penetrator device into the subject. As noted previously, the step of generating a signal may be performed by the computer communicating instructions and/or data to an associated device, for example, visual display, light, auditory device, which then instructs the associated device to produce a signal that can be perceived by the operator.

In one aspect, the computer is configured to generate a visual or auditory signal that alerts the operator to stop further penetration. In one embodiment, a visual display may be configured to generate a visual display such as a “red light” that alerts the operator to stop insertion of the penetrator device. In other embodiments, the computer may transmit instructions to an auditory device, such as a speaker, to produce an auditory signal that alerts the operator to stop penetration of the penetrator device. In some aspects, the computer is configured to generate a visual or auditor signal that alerts the operator to continue insertion of the penetrator device into the subject. In addition, the computer may be configured to generate a signal that alerts the operator to the specific tissue type being imaged by the optical device.

As should be evident to the reader, systems, devices, and methods described herein may be used in a variety of medical procedures where it is desirable to insert a medical device in a subject. For example, the penetrator device and system may be used in procedures such as laparoscopy, thoracoscopy, mediastinoscopy, and insertion of a catheter into a lumen of an anatomical conduit, such as a blood vessel, insertion of a device into solid organs or bony structures, and the like.

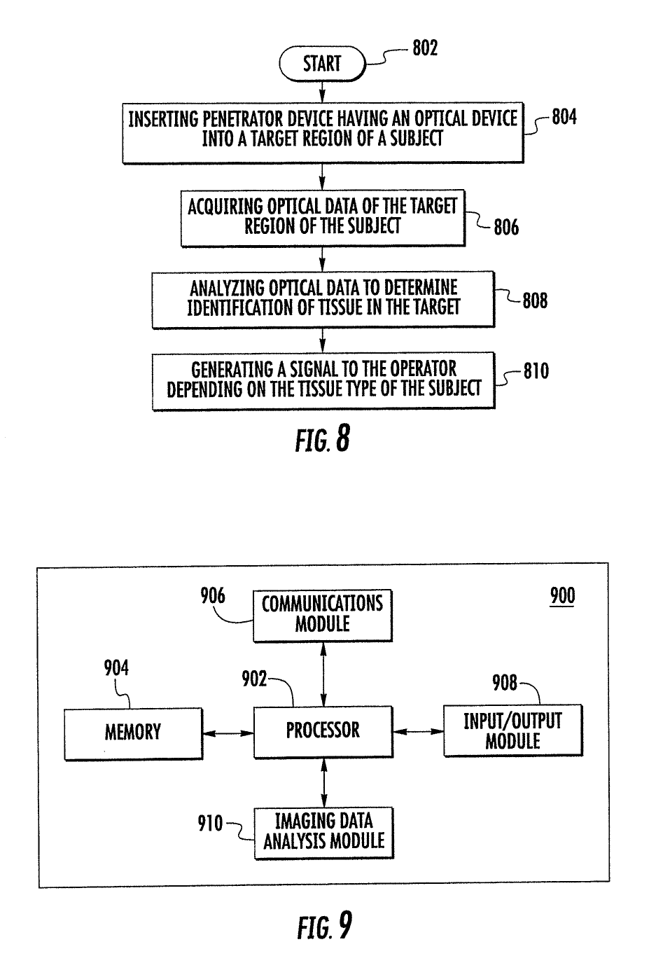

FIG. 9 illustrates a schematic block diagram of circuitry 900, some or all of which may be included in, for example, as part of the imaging system or the computer (see FIGS. 1 and 5, reference characters 14 and 16). As illustrated in FIG. 8, in accordance with some example embodiments, circuitry 900 may include various means, such as a processor 902, memory 904, communication module 906, input/output module 908 and/or imaging data analysis module 910.

As referred to herein, “module” includes hardware, software and/or firmware configured to perform one or more particular functions. In this regard, the means of circuitry 900 as described herein may be embodied as, for example, circuitry, hardware elements (e.g., a suitably programmed processor, combinational logic circuit, and/or the like), a computer program product comprising computer-readable program instructions stored on a non-transitory computer-readable medium (e.g., memory 904) that is executable by a suitably configured processing device (e.g., processor 902), or some combination thereof.

Processor 902 may, for example, be embodied as various means including one or more microprocessors with accompanying digital signal processor(s), one or more processor(s) without an accompanying digital signal processor, one or more coprocessors, one or more multi-core processors, one or more controllers, processing circuitry, one or more computers, various other processing elements including integrated circuits such as, for example, an ASIC (application specific integrated circuit) or FPGA (field programmable gate array), or some combination thereof. Accordingly, although illustrated in FIG. 9 as a single processor, in some embodiments, processor 902 comprises a plurality of processors. The plurality of processors may be embodied on a single computing device or may be distributed across a plurality of computing devices collectively configured to function as circuitry 900. The plurality of processors may be in operative communication with each other and may be collectively configured to perform one or more functionalities of circuitry 900 as described herein. In an example embodiment, processor 902 is configured to execute instructions stored in memory 904 or otherwise accessible to processor 902. These instructions, when executed by processor 902, may cause circuitry 900 to perform one or more of the functionalities of circuitry 900 as described herein.

Whether configured by hardware, firmware/software methods, or by a combination thereof, processor 902 may comprise an entity capable of performing operations according to embodiments of the present invention while configured accordingly. Thus, for example, when processor 902 is embodied as an ASIC, FPGA or the like, processor 902 may comprise specifically configured hardware for conducting one or more operations described herein. As another example, when processor 902 is embodied as an executor of instructions, such as may be stored in memory 904, the instructions may specifically configure processor 902 to perform one or more algorithms and operations described herein.

Memory 904 may comprise, for example, volatile memory, non-volatile memory, or some combination thereof. Although illustrated in FIG. 9 as a single memory, memory 904 may comprise a plurality of memory components. The plurality of memory components may be embodied on a single computing device or distributed across a plurality of computing devices. In various embodiments, memory 904 may comprise, for example, a hard disk, random access memory, cache memory, flash memory, a compact disc read only memory (CD-ROM), digital versatile disc read only memory (DVD-ROM), an optical disc, circuitry configured to store information, or some combination thereof. Memory 904 may be configured to store information, data, applications, instructions, or the like for enabling circuitry 900 to carry out various functions in accordance with example embodiments discussed herein. For example, in at least some embodiments, memory 904 is configured to buffer input data for processing by processor 802. Additionally or alternatively, in at least some embodiments, memory 904 may be configured to store program instructions for execution by processor 902. Memory 904 may store information in the form of static and/or dynamic information. This stored information may be stored and/or used by circuitry 900 during the course of performing its functionalities.

Communications module 906 may be embodied as any device or means embodied in circuitry, hardware, a computer program product comprising computer readable program instructions stored on a computer readable medium (e.g., memory 904) and executed by a processing device (e.g., processor 902), or a combination thereof that is configured to receive and/or transmit data from/to another device, such as, for example, a second circuitry 900 and/or the like. In some embodiments, communications module 906 (like other components discussed herein) can be at least partially embodied as or otherwise controlled by processor 902. In this regard, communications module 906 may be in communication with processor 902, such as via a bus. Communications module 906 may include, for example, an antenna, a transmitter, a receiver, a transceiver, network interface card and/or supporting hardware and/or firmware/software for enabling communications with another computing device. Communications module 906 may be configured to receive and/or transmit any data that may be stored by memory 904 using any protocol that may be used for communications between computing devices. Communications module 906 may additionally or alternatively be in communication with the memory 904, input/output module 908 and/or any other component of circuitry 900, such as via a bus.

Input/output module 908 may be in communication with processor 902 to receive an indication of a user input and/or to provide an audible, visual, mechanical, or other output to a user. Some example visual outputs that may be provided to a user by circuitry 900 are discussed in connection with the displays described above. As such, input/output module 908 may include support, for example, for a keyboard, a mouse, a joystick, a display, an image capturing device, a touch screen display, a microphone, a speaker, a RFID reader, barcode reader, biometric scanner, and/or other input/output mechanisms. In embodiments wherein circuitry 900 is embodied as a server or database, aspects of input/output module 908 may be reduced as compared to embodiments where circuitry 900 is implemented as an end-user machine (e.g., consumer device and/or merchant device) or other type of device designed for complex user interactions. In some embodiments (like other components discussed herein), input/output module 908 may even be eliminated from circuitry 900. Input/output module 908 may be in communication with memory 904, communications module 906, and/or any other component(s), such as via a bus. Although more than one input/output module and/or other component can be included in circuitry 900, only one is shown in FIG. 9 to avoid overcomplicating the drawing (like the other components discussed herein).

Imaging Data Analysis Module 910 may also or instead be included and configured to perform the functionality discussed herein related to analyzing optical data transmitted from the optical system. In some embodiments, some or all of the functionality facilitating analysis of the optical data may be performed by processor 902. In this regard, the example processes and algorithms discussed herein can be performed by at least one processor 902 and/or Imaging Data Module 910. For example, non-transitory computer readable storage media can be configured to store firmware, one or more application programs, and/or other software, which include instructions and other computer-readable program code portions that can be executed to control processors of the components of system 900 to implement various operations, including the examples shown above. As such, a series of computer-readable program code portions may be embodied in one or more computer program products and can be used, with a computing device, server, and/or other programmable apparatus, to produce the machine-implemented processes discussed herein.

Any such computer program instructions and/or other type of code may be loaded onto a computer, processor or other programmable apparatuses circuitry to produce a machine, such that the computer, processor other programmable circuitry that executes the code may be the means for implementing various functions, including those described herein.

The illustrations described herein are intended to provide a general understanding of the structure of various embodiments. The illustrations are not intended to serve as a complete description of all of the elements and features of apparatus, processors, and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

The above disclosed subject matter is to be considered illustrative, and not restrictive, and the appended claims are intended to cover all such modifications, enhancements, and other embodiments, which fall within the true spirit and scope of the description. Thus, to the maximum extent allowed by law, the scope is to be determined by the broadest permissible interpretation of the following claims and their equivalents, and shall not be restricted or limited by the foregoing detailed description.

1. A penetrator device for performing a medical procedure in a subject comprising:
an elongate member having a distal portion and a proximal portion;
a longitudinally extending conduit that extends from the proximal portion of the elongate member to an opening disposed at the distal portion of the elongate member;
a penetrating edge disposed at the distal portion of the elongate member; and
an optical device disposed in the conduit, the optical device being configured to receive optical data of the subject through said opening and to communicate the optical data to an associated processor, wherein the processor is configured to identify the position of the penetrator device within the subject based on the optical data.

2. The device of claim 1, wherein the optical device includes an optical lens and an associated optical fiber.

3. The device of claim 2, wherein the optical lens includes a reflective coating on a surface thereof.

4. The device of claim 1, wherein the device includes a fluid pathway for introducing a gas into the subject via said opening.

5. The device of claim 1, wherein the optical device comprises a longitudinally extending cannula having a distal portion and a proximal portion, an optical lens is disposed in the cannula towards the distal portion thereof, and a fiber optic cable configured to communicate optical data from the optical to lens to the associated processor.

6. A system for inserting a medical device into a subject, the system comprising:
a penetrator device having an elongate member having a distal portion and a proximal portion, a longitudinally extending conduit that extends from the proximal portion of the elongate member to an opening disposed at the distal portion of the elongate member, a penetrating edge disposed at the distal portion of the elongate member, and an optical device disposed in the conduit, the optical device being configured to receive optical data of the subject through said opening;
an optical imaging system in communication with said optical device, the optical imaging system including a light source for emitting light to the optical device;
a fiber optical cable disposed between the optical device and the optical imaging system; and
a processor configured to analyze optical data reflected back to the optical imaging system and identify the position of the penetrator device within the subject based on the optical data.

7. The system of claim 6, wherein the system includes a module that is configured to emit a signal that communicates the position of the device within the subject.

8. The system of claim 6, wherein the system includes a module that is configured to emit a signal that alerts an operator when the device is in a desired position within the subject.

9. The system of claim 7, wherein the signal is visual or auditory.

10. The system of claim 6, wherein the system includes a module that is configured to analyze the intensity of light reflected back to the optical imaging system and determine if the penetrating edge of the penetrator device has entered the peritoneal cavity of the subject.

11. The system of claim 6, wherein the system includes a module that is configured to communicate a signal to instruct an operator to abort the procedure.

12. The system of claim 6, wherein the system includes a module that is configured to detect an index of refraction mismatch in light emitted by the light source.

13. A method of inserting a medical device into a subject, the method comprising:
inserting a medical device into one or more tissue layers of a subject;
electronically collecting optical data of the one or more tissue layers;
analyzing the optical data to determine a position of a penetrating end of the medical device in the subject; and
electronically communicating a signal to an operator identifying the position of the penetrating end of the medical device in the subject.

14. The method of claim 13, wherein the step of electronically communicating a signal comprises generating a visual or auditor signal.

15. The method of claim 13, further comprising the step of electronically generating a signal that alerts the operator if the penetrating end of the medical device is in a desired position in the subject.

16. The method of claim 13, further comprising the step of electronically generating a signal that alerts the Operator if the penetrating end of the medical device is in an undesired position in the subject.

17. The method of claim 13, further comprising the step of electronically generating a signal that alerts the Operator to continue the step of inserting the medical device.

18. The method of claim 13, further comprising the step of electronically generating a signal that alerts the operator to abort the stop of inserting the medical device.

19. The method of claim 13, wherein the step of electronically communicating a signal comprises generating a green light to alert the operator to continue to insert the medical device.

20. The method of claim 13, wherein the step of electronically communicating a signal comprises generating a red light to alert the Operator to stop the step of inserting the medical device.

21. The method of claim 13, wherein the step of electronically communicating a signal further comprises identifying a tissue type of the subject that is adjacent to the penetrating end of the medical device.
